(19) United States
(12) Patent Application Publication
(10) Pub. No.: US 2023/0240666 A1
(43) Pub. Date: Aug. 3, 2023

(54) SAMPLE COLLECTION DEVICE

(71) Applicant: Quanta Vision Co., Ltd., Taipei City (TW)

(72) Inventor: Ya-Chieh YU, Taipei City (TW)

(21) Appl. No.: 17/649,772

(22) Filed: Feb. 2, 2022

(51) Int. Cl. A61B 10/00; B01L 3/00

(52) U.S. Cl. CPC A61B 10/0051; B01L 3/5029

(57) ABSTRACT

A sample collection device includes a main body. The main body includes a collection member and a protection member. The protection member is umbrella-shaped and disposed on the main body and one side of the collection member. The protection member includes a support frame, a canopy and a seal portion. The support frame is openable and closable relative to the main body to selectively hold the canopy in an open state and close the canopy in a closed state can be opened or closed relative to the base.

### BACKGROUND OF THE INVENTION

### Field of the Invention

The present invention relates to a sample collection device.

### Description of the Prior Art

Today there are many prevalent infectious diseases transmitted through droplets such as covid-19, influenza and tuberculosis. In order to prevent the rapid spread of infectious diseases, many fast testing stations and home test kits are provided for self-testing or assistance testing. Generally, the diseases are collected from an oral or a nasal cavity, which may cause an examiner to be at risk of infection.

TWM615921 discloses a movable collapsible sample collection kiosk which provides a partition to separate the examiner and an examinate on opposite two sides thereof so as to reduce the risk of infection by reducing direct contact between the examiner and the examinate. However, a size of the movable collapsible sample collection kiosk is too large to carry. Thus, the movable collapsible sample collection kiosk is only suitable for medical institutions or companies, but not suitable for self-testing.

The present invention is, therefore, arisen to obviate or at least mitigate the above-mentioned disadvantages.

### SUMMARY OF THE INVENTION

The main object of the present invention is to provide a sample collection device, which can block droplets via a protection member during collection of a sample and is convenient to carry.

To achieve the above and other objects, the present invention provides a sample collection device, including: a main body, including a collection member; and a protection member, being umbrella-shaped, disposed on the main body and one side of the collection member, including a support frame, a canopy and a seal portion, the support frame disposed on the canopy, the support frame being openable and closable relative to the main body to selectively hold the canopy in an open state and close the canopy in a closed state, the canopy has a through hole disposed therethrough, and the seal portion circumferentially disposed around and between an edge of the through hole and the main body.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of a first preferable embodiment of the present invention;

FIG. 2 is a breakdown drawing of the first preferable embodiment of the present invention;

FIG. 3 is a drawing showing a canopy of the first preferable embodiment of the present invention in an open state;

FIG. 4 is a partial cross sectional view of the first preferable embodiment of the present invention;

FIG. 4A is a partially-enlarged view of FIG. 4;

FIG. 5 is a drawing showing the first preferable embodiment of the present invention in use;

FIG. 6 is a stereogram of a canopy of a second preferable embodiment of the present invention in an open state; and

FIG. 7 is a stereogram of a canopy of the second preferable embodiment of the present invention in a closed state.

### DETAILED DESCRIPTION OF THE PREFERRED EMBODIMENTS

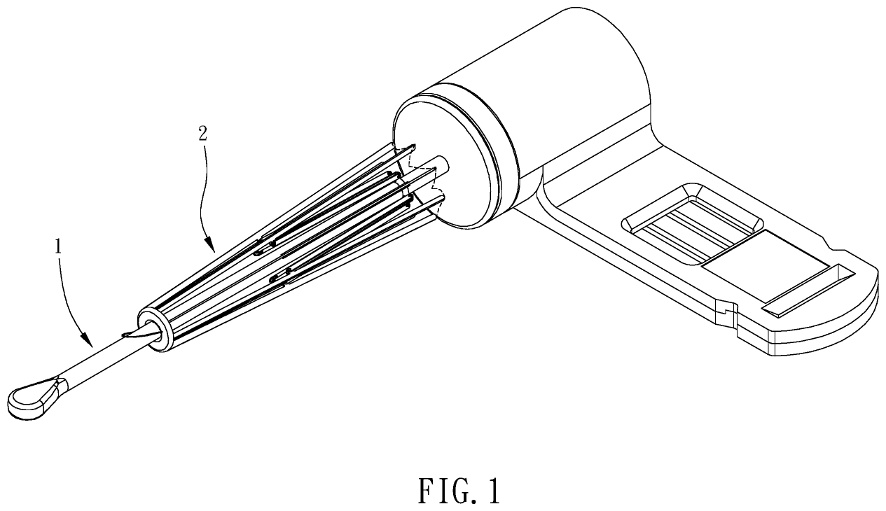

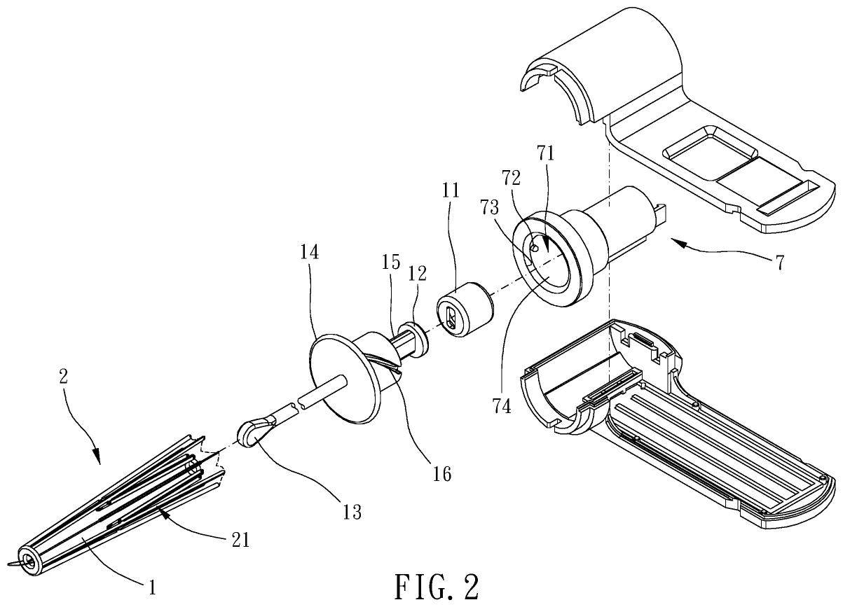

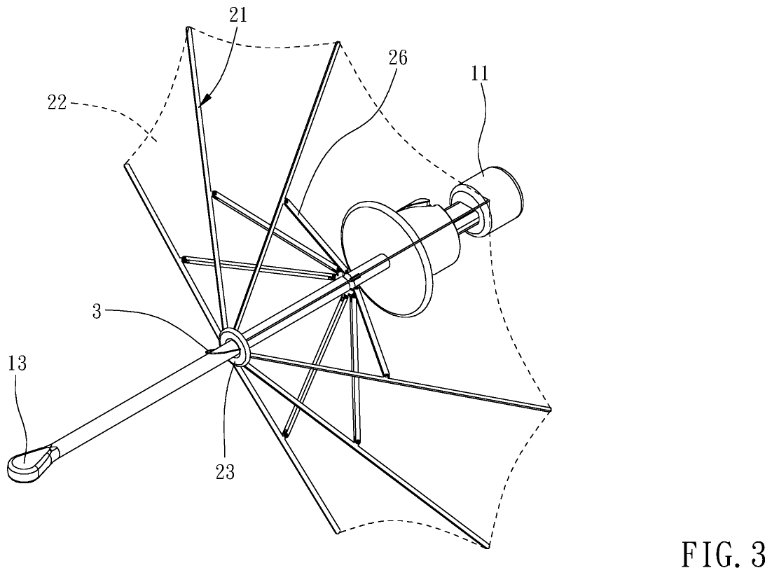

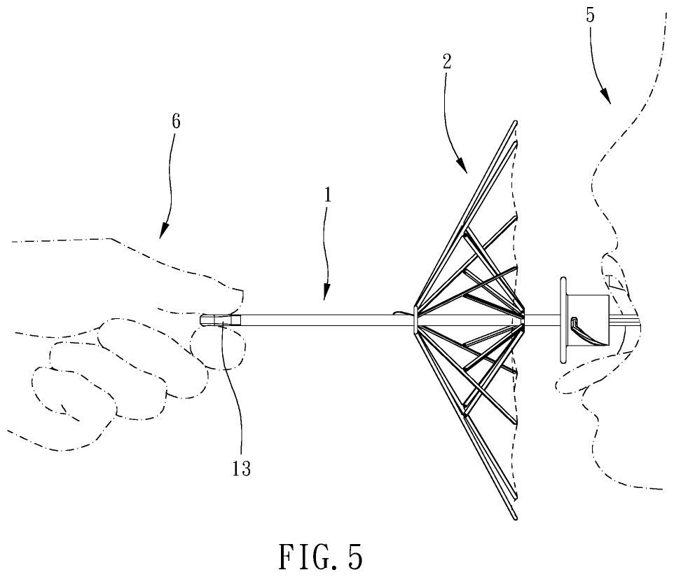

Please refer to FIGS. 1 to 5 for a first preferable embodiment of the present invention. A sample collection device of the present invention includes a main body 1 and a protection member 2.

The main body 1 includes a collection member 11 which is made of material having ability or tendency to absorb liquid, such as cotton, sponge, rayon and polyester, so as to easy collect a sample (such as a saliva sample, a respiratory tract sample and other samples) from an examinate 5. The protection member 2 is umbrella-shaped. The protection member 2 is disposed on the main body 1 and one side of the collection member 11. The protection member 2 includes a support frame 21, a canopy 22 and a seal portion 23. The canopy 22 can be a waterproof cloth. The support frame 21 is disposed on the canopy 22. The support frame 21 is openable and closable relative to the main body 1 to selectively hold the canopy 22 in an open state and close the canopy 22 in a closed state. The canopy 22 has a through hole 221 disposed therethrough. The seal portion 23 is circumferentially disposed around and between an edge of the through hole 221 and the main body 1, so that the seal portion 23 can block the liquid. Therefore, when an examiner 6 collects the sample from the examinate 5, the protection member 2 can block the droplets. Thus, it provides a high protection effect. Moreover, the protection member 2 can be closed to small size so that it is convenient to carry and can save space.

The canopy 22 is a deformable and transparent canopy, so that the examiner 6 can directly view the examinate 5 through the canopy 22 for easy collecting the sample from the examinate 5.

The seal portion 23 is made of silicone rubber or rubber, which provides anti-slip effect and high sealing effect. Thus, the seal portion 23 is easy positioning.

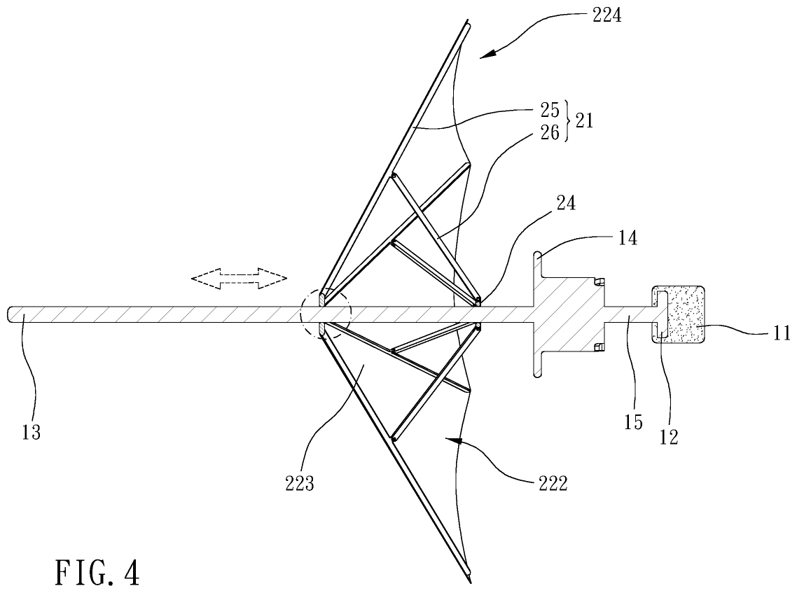

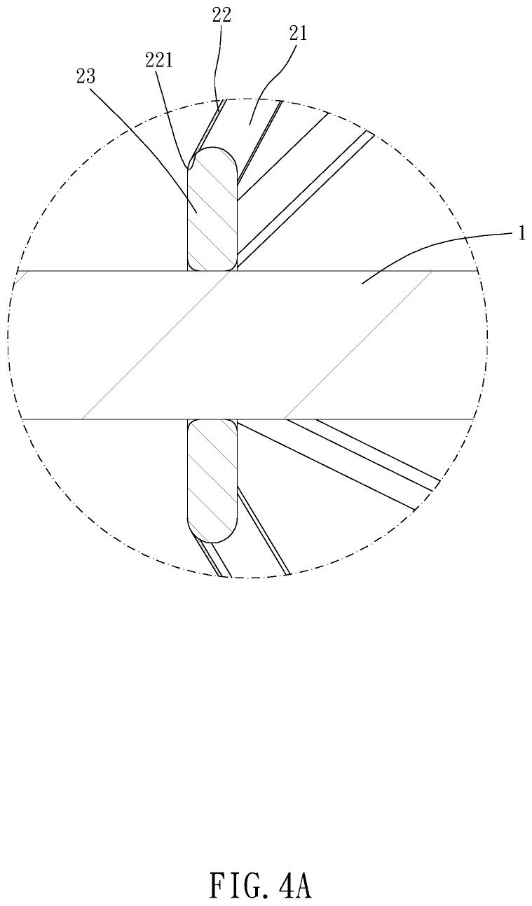

The support frame 21 includes a positioning member 24, a support frame portion 25 and a connection frame portion 26. The seal portion 23 is a seal ring. The support frame portion 25 is connected between the canopy 22 and the seal portion 23. In this embodiment, one end of the support frame portion 25 is pivoted to the seal portion 23. The positioning member 24 is disposed on the main body 1 and is located between the collection member 11 and the seal portion 23. The connection frame portion 26 is movably connected between the positioning member 24 and the support frame portion 25. The seal portion 23 is movable relative to the main body 1 between a first position and a second position to move the canopy 22 and the support frame portion 25. Wherein when the seal portion 23 is in the first position, the connection frame portion 26 and the support frame portion 25 are respectively opened relative to the main body 1 to spread the canopy 22 in the open state (as shown in FIG. 4). When the seal portion 23 is in the second position, the connection frame portion 26 and the support frame portion 25 are respectively closed relative to the main body 1 to close the canopy 22 in the closed state (as shown in FIG. 1). Preferably, the main body 1 includes a connection portion 12 and a handle portion 13. The collection member 11 is disposed on the connection portion 12. The support frame portion 25 is connected with the seal portion 23 and extends from the seal portion 23 toward the collection member 11. The protection member 2 is disposed between the connection portion 12 and the handle portion 13. The second position is closer to the handle portion 13 than the first position. Preferably, the sample collection device further includes a pull member 3. The pull member 3 can be a ring body or a rope. The pull member 3 is disposed on the protection member 2 and is at least partially exposed on a side of the protection member 2 facing the handle portion 13. The pull member 3 is movable relative to the main body 1 to move the seal portion 23 to move relative to the main body 1, so as to easy open or close the protection member 2. Furthermore, it can be reduced a risk of infection during operation of opening or closing the protection member 2. Specifically, when the examiner 6 operates to close or open the protection member 2, the examiner 6 and the examinate 5 are on opposite two sides of the protection member 2, so that the examiner 6 is not easy to be infected.

It is noted that, when the canopy 22 is in the open state, a shield surface 222 of the canopy 22 faces the collection member 11. When the canopy 22 is in the closed state, the shield surface 222 of the canopy 22 faces the main body 1. The shield surface 222 is concave and is formed a recess 223 recessed away from the collection member 11, and an opening 224 of the recess 223 opens toward the collection member 11. Therefore, the droplets of the examinate 5 can be blocked by the shield surface 222 and collected within the recess 223, so that the droplets do not drip easily. Thus, it can reduce the risk of infection. Moreover, hands can avoid touching the shield surface 222 during operation of closing the canopy 22. When the canopy 22 is in the closed state, the shield surface 222 can be directly folded inside of the canopy 22 and the droplets on the shield surface 222 can be covered inside the canopy 2.

The main body 1 further includes a retaining flange 14 which extends radially. The retaining flange 14 is disposed between the through hole 221 and the collection member 11. An outer diameter size of the retaining flange 14 is respectively greater than an inner diameter size of the through hole 221 and an outer diameter size of the collection member 11, so that the droplets can be blocked by the retaining flange 14. Thus, the droplets are not easy to splash through the through hole 221.

The main body 1 further includes a connection section 15 which is connected between the connection portion 12 and the retaining flange 14. Therefore, the collection member 11 can respectively keep the retaining flange 14 and the protection member 2 in a distance, so as to prevent the examinate 5 from being touched by the protection member 2. Moreover, the collection member 11 can be placed deep into a mouth or a nasal cavity of the examinate 5.

The sample collection device further includes a container 7. The container 7 includes a receiving recess 71. One of an outer surface of the main body 1 and an inner wall 74 of the receiving recess 71 of the container 7 includes at least one spiral guiding slot 16, the other of the outer surface of the main body 1 and the inner wall 74 of the receiving recess 71 of the container 7 includes at least one protruding structure 72. The at least one protruding structure 72 is slidably engaged within the at least one spiral guiding slot 16. The collection member 11 is insertable into the receiving recess 71. When the canopy 22 is in the open state, an outer diameter size of the canopy 22 is greater than an inner diameter size of a notch 73 of the receiving recess 71. When the collection member 11 is rotatably axially moved to squeeze the inner wall 74 of the receiving recess 71, the canopy 22 can block the sample which is splashed out of the receiving recess 71.

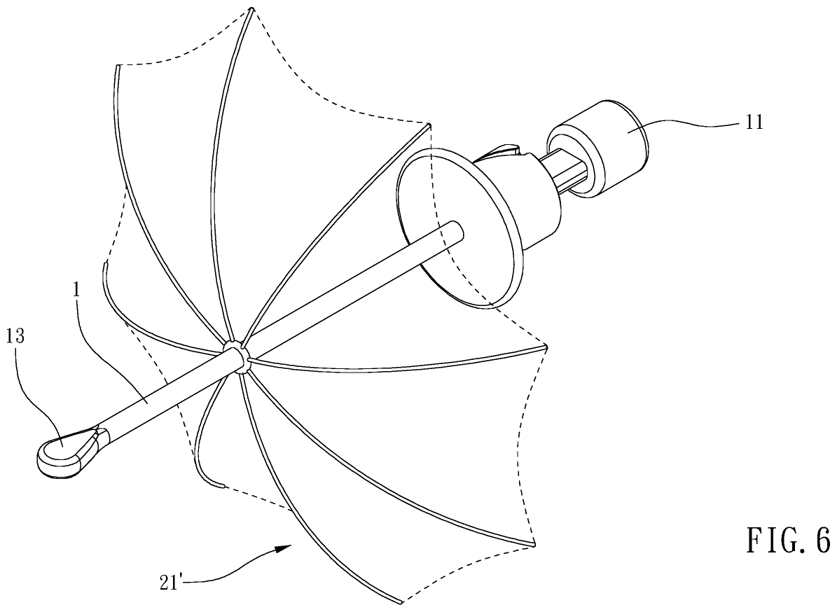

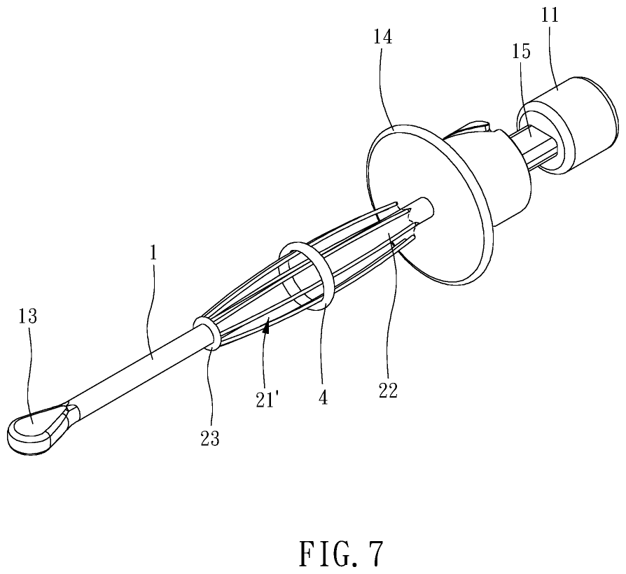

Please refer to FIGS. 6 to 7 for a second preferable embodiment of the present invention. The support frame 21′ is made of deformable material, such as rubber and silicone rubber. The support frame 21′ is normally elastically spread the canopy 22 to keep the canopy 22 in the open state. The support frame 21′ is deformable by pressure to move toward the main body 1 to close the canopy 22 in the closed state. In this embodiment, the support frame 21′ is integrally connected with the seal portion 23, so as to easy manufacture.

Preferably, the sample collection device further include a retaining sleeve 4 which is removably sleeved on the protection member 2, so as to keep the canopy 22 in the closed state, thus the droplets can be covered inside the canopy 22.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A sample collection device, including:
a main body, including a collection member; and
a protection member, being umbrella-shaped, disposed on the main body and one side of the collection member, including a support frame, a canopy and a seal portion, the support frame disposed on the canopy, the support frame being openable and closable relative to the main body to selectively hold the canopy in an open state and close the canopy in a closed state, the canopy has a through hole disposed therethrough, and the seal portion circumferentially disposed around and between an edge of the through hole and the main body.

2. The sample collection device of claim 1, wherein the support frame includes a positioning member, a support frame portion and a connection frame portion, the seal portion is a seal ring, the support frame portion is connected between the canopy and the seal portion, the positioning member is disposed on the main body and is located between the collection member and seal portion, the connection frame portion is movably connected between the positioning member and the support frame portion, the seal portion is movable relative to the main body between a first position and a second position to move the canopy and the support frame portion; wherein when the seal portion is in the first position, the connection frame portion and the support frame portion are respectively opened relative to the main body to spread the canopy; and when the seal portion is in the second position, the connection frame portion and the support frame portion are respectively closed relative to the main body.

3. The sample collection device of claim 2, wherein the main body includes a connection portion and a handle portion, the collection member is disposed on the connection portion, the support frame portion is connected with the seal portion and extends from the seal portion toward the collection member, the protection member is disposed between the connection portion and the handle portion, and the second position is closer to the handle portion than the first position.

4. The sample collection device of claim 3, further including a pull member, the pull member disposed on the protection member and being at least partially exposed on a side of the protection member facing the handle portion, and the pull member being movable relative to the main body to move the seal portion to move relative to the main body.

5. The sample collection device of claim 1, wherein the support frame is made of deformable material, the support frame is normally elastically spread the canopy to keep the canopy in the open state, and the support frame is deformable by pressure to move toward the main body.

6. The sample collection device of claim 1, wherein the seal portion is made of silicone rubber or rubber.

7. The sample collection device of claim 1, wherein when the canopy is in the open state, a shield surface of the canopy faces the collection member; and when the canopy is in the closed state, the shield surface of the canopy faces the main body.

8. The sample collection device of claim 7, wherein the shield surface is concave and is formed a recess recessed away from the collection member, and an opening of the recess opens toward the collection member.

9. The sample collection device of claim 4, wherein the canopy is a deformable and transparent canopy; the seal portion is made of silicone rubber or rubber; when the canopy is in the open state, a shield surface of the canopy faces the collection member; and when the canopy is in the closed state, the shield surface of the canopy faces the main body; the shield surface is concave and is formed a recess recessed away from the collection member, and an opening of the recess opens toward the collection member; the main body further includes a retaining flange which extends radially, the retaining flange is disposed between the through hole and the collection member, an outer diameter size of the retaining flange is respectively greater than an inner diameter size of the through hole and an outer diameter size of the collection member; the main body further includes a connection section which is connected between the connection portion and the retaining flange; the sample collection device further includes a container, the container includes a receiving recess, one of an outer surface of the main body and an inner wall of the receiving recess of the container includes at least one spiral guiding slot, the other of the outer surface of the main body and the inner wall of the receiving recess of the container includes at least one protruding structure, the at least one protruding structure is slidably engaged within the at least one spiral guiding slot, the collection member is insertable into the receiving recess; wherein when the canopy is in the open state, an outer diameter size of the canopy is greater than an inner diameter size of a notch of the receiving recess.

10. The sample collection device of claim 5, wherein the support frame is integrally connected with the seal portion; the canopy is a deformable and transparent canopy; when the canopy is in the open state, a shield surface of the canopy faces the collection member; and when the canopy is in the closed state, the shield surface of the canopy faces the main body; the shield surface is concave and is formed a recess recessed away from the collection member, and an opening of the recess opens toward the collection member; the main body further includes a retaining flange which extends radially, the retaining flange is disposed between the through hole and the collection member, an outer diameter size of the retaining flange is respectively greater than an inner diameter size of the through hole and an outer diameter size of the collection member; the main body further includes a connection section which is connected between the connection portion and the retaining flange; the sample collection device further includes a retaining sleeve which is removably sleeved on the protection member to keep the canopy in the closed state; the sample collection device further includes a container, the container includes a receiving recess, one of an outer surface of the main body and an inner wall of the receiving recess of the container includes at least one spiral guiding slot, the other of the outer surface of the main body and the inner wall of the receiving recess of the container includes a protruding structure, the at least one protruding structure is slidably engaged within the at least one spiral guiding slot, the collection member is insertable into the receiving recess; wherein when the canopy is in the open state, an outer diameter size of the canopy is greater than an inner diameter size of a notch of the receiving recess.
